(19) United States
(12) Patent Application Publication
(10) Pub. No.: US 2025/0345193 A1
(43) Pub. Date: Nov. 13, 2025

(54) Piezoelectric Stents with Self-Powered Anti-Restenosis Properties

(71) Applicant: Wisconsin Alumni Research Foundation, Madison, WI (US)

(72) Inventors: Xudong Wang, Middleton, WI (US); Bo Liu, Madison, WI (US); Fengdan Pan, Madison, WI (US); Zulmari Silva-Pedraza, Madison, WI (US); Jack Bontekoe, Fitchburg, WI (US)

(21) Appl. No.: 18/660,385

(22) Filed: May 10, 2024

(51) Int. Cl. A61F 2/91; A61F 2/30; A61L 31/16

(52) U.S. Cl. CPC A61F 2/91; A61L 31/16; A61F 2002/30985

(57) ABSTRACT

An electric field-assisted 3D printing system that allows for fast printing of complex and spontaneously polarized ferroelectric structures with high fidelity and superb piezoelectric performance. The system provides the basis for development of a piezoelectric vascular structure that can serve as a stent, providing self-powered electricity to prevent restenosis and biologic growth by producing a low intensity electric field around the stent.

### STATEMENT REGARDING FEDERALLY SPONSORED RESEARCH OR DEVELOPMENT

This invention was made with government support under HL157077 awarded by the National Institutes of Health. The government has certain rights in the invention.

### BACKGROUND OF THE INVENTION

The present invention relates to a 3D printed piezoelectric material suitable for use as a transducer, and more particularly, a stent with piezoelectric response for producing a low intensity electric field driven by changes in vessel diameter and blood flow.

Stenting is a common strategy to treat cardiovascular diseases, such as peripheral arterial disease and coronary artery disease, as well as colonic strictures and ureteral obstructions. Implantable stents are typically made from ultrafine stainless steel wires weaved into a zigzag network, which provides mechanical support to widen and open the lumen of anatomic vessels to maintain the circulation of blood. Stents also help provide the backbone support of synthetic tubing and are known as covered stents and stent grafts, which can be used to re-line the inside of weakened blood vessels. Stents are typically delivered to the target by catheters that are minimally invasive and then expanded to support the blood vessels. Based on the mechanism of expanding, stents are designed to be self-expandable or balloon expanded. After deployment, stents are normally kept on site within the blood vessel over the patient's lifetime, expanding and contracting with the blood vessels and facing dynamic blood flow together with pressure fluctuations.

Peripheral arterial disease (PAD) develops when atherosclerotic plaque builds up in arteries that provide blood supply to the limbs, brain, kidney, and other organs. Approximately 200 million individuals in the world have PAD. Autopsy studies have demonstrated that when subclinical disease is considered, PAD is more prevalent than coronary heart disease. The arteries in the lower limbs (such as femoropopliteal artery) are the most common sites of PAD, typically characterized by multilevel steno-occlusive disease with complex calcified morphology. PAD symptoms range from leg pain when walking (claudication) to non-healing wounds on toes, feet or legs. If left untreated or inadequately treated, PAD may become disabling and life-threatening.

Balloon angioplasty and stenting are the primary methods of endovascular revascularization for patients with severe PAD symptoms. However, the 2-year patency rate of angioplasty and bare-metal stenting in the lower extremities is only 50-70%. Stent failure occurs due to pathologic processes such as restenosis or thrombosis, which block the lumen of the stent, infection, or stent fracturing.

Drug-eluting stents-first approved by FDA in 2003—are widely viewed as a transformative treatment for coronary atherosclerosis. These drug-eluding stents coat the existing bare-metal stents with drugs that slow release to prevent blood clots. However, the same approach has limited success in PAD patients. For example, controlled clinical trials showed all-cause death at 2-years was significantly increased in PAD patients treated with paclitaxel-coated stents versus those treated with bare-metal stents (7.2% versus 3.8%; the paclitaxel-associated mortality rate with a 5-year follow-up increased further (14.7% versus 8.1%)). The exact mechanism of higher mortality in PAD patients treated with drug-eluting stents is unknown, however, meta-regression analysis showed a significant correlation between paclitaxel exposure (dose×time) and mortality.

Therefore, there remains a need for safe and dependable long-term stents for PAD patients.

### SUMMARY OF THE INVENTION

To safely reduce restenosis and stent failure, the present inventors have invented a piezoelectric biomaterial to generate a low-intensity electric field around the stent. The low-intensity electric field can suppress biologic growth which is the initial step of biofouling. Thus, piezoelectric stents are an effective technique in preventing cell attachment therefore inhibiting restenosis and infection. The present invention is applicable to most implantable vascular devices to prevent occlusion, thrombosis or biofouling.

Recent advancements in biocompatible piezoelectric materials have enabled in vivo coupling between biomechanical motions and localized electricity generation in biological systems. The incorporation of biocompatible piezoelectric materials to biomedical devices offers unprecedented functionalities for energy harvesting, drug delivery, actuators and physiological sensing. Developing stents using biocompatible piezoelectric materials opens new opportunities to introduce self-sustainable in vivo electricity generation, leading toward multi-functionality in future stent technology.

The present inventors have designed and fabricated piezoelectric stents using fused deposition modeling (FDM) 3D printing with in situ poling. In certain embodiments, the stents are designed with a zigzag shape made from composite wires of potassium sodium niobate (KNN) particles and poly (vinylidene fluoride-co-hexafluoropropylene) (PVDF-HFP). The stents show desired mechanical properties and piezoelectric property. Therefore, they can produce appreciable piezoelectricity when subject to mechanical strains, thus providing electricity generation driven by regular blood pressure and mechanical fluctuation.

The target environment within blood vessels places the piezoelectric stent at a unique position to constantly interface with biomechanical agitations. If utilized effectively, regular blood pressure and mechanical fluctuation may provide an endless energy source to support local and in vivo electronic functionalities, such as sensing, electrical stimulation, drug delivery, tissue healing or anti-thrombosis.

In certain embodiments, the stents are constructed of a piezoelectric biomaterial formed into a micro lattice and produce desired electrical pulses based on mechanical changes in blood vessel pulsations, blood flow, and pressures. The low electric potential generated by the stent material has anti-stenotic and anti-microbial properties and does not cause long term harm to the cellular components of the arterial wall.

One embodiment of the present invention provides a stent comprising a tube defined by a piezoelectric substrate material formed in a cylindrical lattice pattern permitting compression and expansion of the tube; wherein the piezoelectric substrate is poled with respect to at least one axis of the tube; and wherein the piezoelectric substrate generates a voltage of at least 10 mV under pressure changes on an inside or outside of the tube to create an electric field around the tube.

It is thus one feature of at least one embodiment of the present invention to solve stent failure in PAD patients in a different manner from the drug-eluting stents by providing a piezoelectric biomaterial that can generate low-intensity alternative electric fields in response to blood pressure and mechanical fluctuation to disturb the inner and outer surface of the stent.

The piezoelectric substrate can generate a peak-to-peak voltage output of 10-150 mV under pressure changes between 10 mmHg to 40 mmHg on the inside or outside of the tube. The piezoelectric substrate can generate a voltage of at least 10 mV under pressure changes of at least 40 mmHg on the inside or outside of the tube. The electric field may be less than 12 V/cm.

It is thus one feature of at least one embodiment of the present invention to produce low-intensity piezoelectricity self-generated by the stents under normal pressures changes within the body to inhibit cell attachment on the stent surface.

The piezoelectric substrate may be ferroelectric potassium sodium niobite (KNN) particles embedded in a ferroelectric polyvinylidene fluoride (PVDF) polymer matrix. The substrate may be poled in a radial direction or an axial direction of the tube.

It is thus one feature of at least one embodiment of the present invention to provide strong piezoelectric composite materials that may be 3D printed and poled simultaneously without losing mechanical integrity.

The piezoelectric substrate may have a thickness of less than or equal to 250 μm. The tube may have a diameter between 2 mm to 50 mm or between 2 to 20 mm or about 6 mm or about 2.5 mm. The piezoelectric substrate may vary in length and may have a range between 2 to 200 mm.

It is thus one feature of at least one embodiment of the present invention to produce a piezoelectric tube with uniform diameter and thickness to produce uniform electric field around the stent according to the size of the target vessel, but also reduce the hemodynamic and mechanical disturbances to the vessel wall.

The piezoelectric substrate may be a lattice of zigzag rings joined by inclined bridges.

It is thus one feature of at least one embodiment of the present invention to allow the stent to expand and contract with blood pressure and mechanical fluctuations of the vessels without breaking and to produce an electrical charge through mechanical movement.

One embodiment of the present invention provides a method of manufacturing a stent comprising a tube defined by a substrate of piezoelectric material formed in a cylindrical lattice permitting compression and expansion of a diameter of the tube wherein the substrate is poled with respect to at least one axis of the substrate, the method comprising the steps of: heating a composite piezoelectric material at a temperature of at least 250 degrees Celsius to form a molten material; extruding the molten material through a 3D printer nozzle; applying an electrical field between the 3D printer nozzle and a stainless steel rod to pole the molten material as the molten material is being extruded; depositing the molten material onto the stainless steel rod along an axis of the rod; and rotating the rod as the molten material is deposited to form the substrate of piezoelectric material into a tube.

It is thus one feature of at least one embodiment of the present invention to provide an innovative technique for 3D-printing of piezoelectric biomaterials for stent fabrication by building the stent directly on a rotating stainless steel rod.

The stainless steel rod may have a diameter between 2 mm to 50 mm or between 2 to 20 mm or about 6 mm or about 2.5 mm.

It is thus one feature of at least one embodiment of the present invention to allow the diameter of the stainless steel rod to control the diameter of the stent.

The stainless steel rod may be rotated one rotation every 5 seconds. The molten material may be deposited at a print speed of less than 20 mm/s.

It is thus one feature of at least one embodiment of the present invention to allow for simultaneous poling of the molten material as the tubular substrate is being extruded.

An adhesive layer may be applied to the stainless steel rod prior to depositing the molten material onto the stainless steel rod.

It is thus one feature of at least one embodiment of the present invention to promote adhesion of the molten material as the stainless steel is rotated.

One embodiment of the present invention provides a method of stenting an anatomical vessel to prevent restenosis, the method comprising the steps of providing a stent defined by a tube having a substrate of piezoelectric material formed in a cylindrical lattice permitting compression and expansion of the tube wherein the substrate is poled with respect to at least one axis of the substrate; inserting the stent into a lumen of the anatomical vessel; and generating a peak-to-peak voltage output in response to anatomical vessel fluctuations of at least 10 mV under pressure changes of less than 40 mmHg on an inside or outside of the stent to create an electric field around the stent.

It is thus one feature of at least one embodiment of the present invention to transform intravascular devices and use the design and concept on other implanted devices such as pacemakers, synthetic blood vessels or other encapsulated devices implanted in the body.

The method may further comprise creating an alternating positive or negative voltage output based on the anatomical vessel fluctuations. The method may further comprise creating an alternating electrical field based on the anatomical vessel fluctuations.

It is thus one feature of at least one embodiment of the present invention to produce self-generated piezoelectric pulses that can effectively suppress biologic attachment or growth on vascular devices.

The electric field strength may be less than 12 V/cm. The method may further comprise disturbing the inner and outer surfaces of the tube to prevent biologic growth.

It is thus one feature of at least one embodiment of the present invention to rely upon electrical double layer disturbance the on poled substrate, which acts locally on the stent surface to suppress biologic formation. The electric field strength is low enough to not harm the nearby cells of the blood vessel wall and tissues.

These particular objects and advantages may apply to only some embodiments falling within the claims and thus do not define the scope of the invention.

### BRIEF DESCRIPTION OF THE DRAWINGS

FIG. 1 is a schematic representation of one embodiment of the present invention showing a method of printing a piezoelectric substrate tube where it is shown, in the enlarged inset, that the molten polymer composite is poled normal to the substrate simultaneous with 3D printing extrusion to form the piezoelectric substrate tube on a rotating stainless steel rod;

FIG. 2 is a schematic representation of an alternative embodiment of the present invention showing a method of printing a planar piezoelectric substrate on a build plate where the molten polymer composite is poled in-plane by interdigitated poling electrodes simultaneous with 3D printing extrusion to form the piezoelectric substrate which is subsequently rolled into the piezoelectric substrate tube;

FIG. 3 is an enlarged perspective view of one embodiment of the piezoelectric substrate tube formed by the method of FIG. 1 having zigzag rings joined by diagonal bridges and responding to blood pressure to produce a voltage creating a low intensity electric field around the stent;

FIG. 4 is a schematic representation of a cross section of the wall of the poled piezoelectric substrate tube of FIG. 3 responding to blood pressure to produce alternating low intensity electric field that disrupts the surfaces of the stent; and

FIG. 5 is a schematic representation of one embodiment of the piezoelectric substrate tube implanted within a blood vessel causing surface electrical double layer agitation or disturbance to suppress biologic growth.

### DETAILED DESCRIPTION OF THE PREFERRED EMBODIMENT

### Background

The pathophysiology of restenosis involves vascular smooth muscle cells (SMCs)—the major cell type residing in the arterial wall. Experimental data demonstrates that SMCs in the arterial wall regain the ability to proliferate and migrate in response to vessel injuries caused by angioplasty and stenting.

Using polymers with slow-release properties, device manufacturers modified the existing stent technology by coating bare-metal stents with paclitaxel or other chemotherapeutic drugs. This approach allows sustained and local accumulation of relatively higher concentrations of drugs that block SMC proliferation and migration. However, there are several drawbacks to this approach particularly when treating PAD.

First, drugs released from the stents inhibit proliferation and migration of all cell types in the blood vessels including vascular endothelial cells that are necessary for repairing injured endothelium. As healthy endothelium is an anti-thrombotic barrier, delayed thrombosis is a major side effect of drug eluting stents.

Second, long term systematic effects of drugs released from the stents are a concern particularly in PAD patients. Because of the size of peripheral arteries and the diffused nature of atherosclerotic plaques built up in the periphery, stents used to treat PAD are significantly larger in diameter and length than the stents used in coronary patients. It has been suggested that the amount of paclitaxel leaked to the circulation of patients treated with drug-eluting stents is, at least in part, responsible for the elevated mortality.

Electricity has been studied as a promising physical approach to prevent the attachments of biological species. For example, electrochemical sterilization typically uses locally generating biocides (e.g., reactive oxygen species) via electrochemical water electrolysis or by immobilization of electro-generated biocides. Additionally, high frequency (up to 1000 Hz), strong electric fields (1-100 kV/cm) have also been shown to kill microbes by irreversible electroporation of cell membranes or to repel microbes via the dielectrophoretic effect. These electrical strategies, though generally very effective, are complex for implementation and have very strict application conditions, thus are not suitable for small implanted systems.

The present inventors have discovered a new mechanism for electronically preventing the attachment of biological species. Introduction of a low frequency, alternating electric field (˜12V/cm) to a pair of working electrode leads on a glass substrate prevented attachment of microbes contained in natural lake water to the area between the two electrodes. It is known that in a liquid environment, an electrical double layer will form at the surface, providing electrostatic force to attract organic substances such as proteins toward the surface. The presence of a weak and low frequency, alternating electric field can create an unstable distribution of the electrical double layer. As such, the ion distribution in the double layer fluctuates as the electric field oscillates back and forth, which subsequently disrupts the electrostatic force and thus the organic coating. Without a stable organic coating, the microbes cannot bind and achieve strong adhesion to the material surface. Fluid shear forces can then easily remove these microbes.

The above described mechanism is described in “Effective anti-biofouling enabled by surface electric disturbance from water wave-driven nanogenerator,” found at https://doi.org/10.1016/j.nanoen.2018.12.069, hereby incorporated by reference.

A similar physical approach can be applied to the present invention as an effective and safe way to prevent SMCs attachment to implanted stents.

### The Present Invention

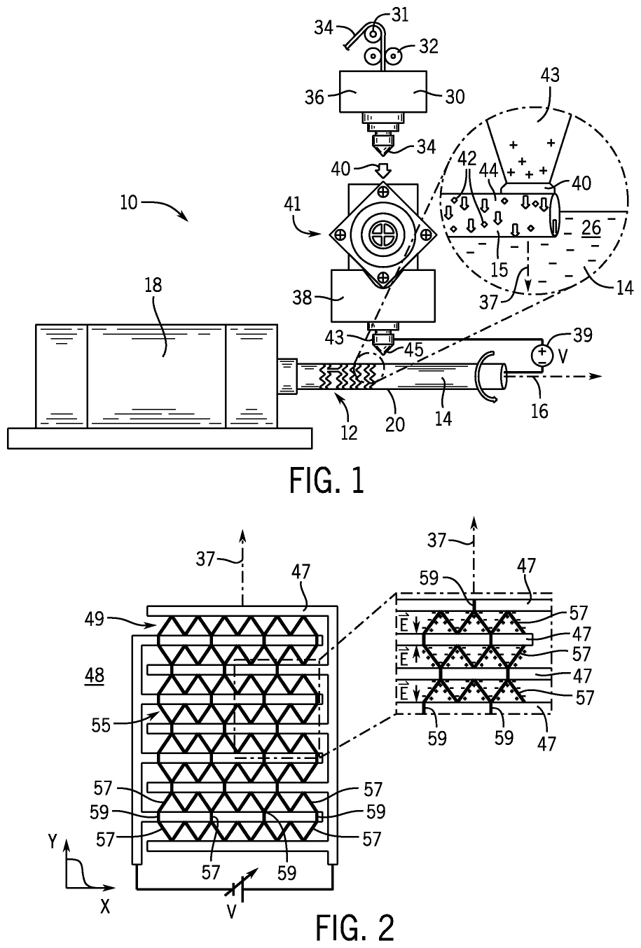

Referring to FIG. 1, a three-dimensional (3D) printing system 10 of the present invention for fused deposition modeling (FDM) may be used with an electric poling process in the manufacture of piezoelectric substrate tube 12, for example, used as stents within the body.

The 3D printing system 10 may include a stainless steel rod 14 providing a cylinder rotating about a longitudinal axis 16 by a gear motor 18. The stainless steel rod 14 may extend substantially horizontally in cantilever from the box of the gear motor 18. In one embodiment, the stainless steel rod 14 may have a diameter between 2 mm to 50 mm or between 2 to 20 mm or about 6 mm or about 2.5 mm and may be driven by a 12 V gear motor. The gear motor 18 may be any type of electric motor for enabling rotation of the stainless steel rod 14 as understood in the art.

The stainless steel rod 14 has a curved outer surface 20 forming a circumference about the longitudinal axis 16 on which the extruded molten material 15 is deposited directly during the 3D printing process to produce a cylindrical substate that has substantially the same diameter as the stainless steel rod 14. Thus, the diameter of the stainless steel rod 14 may vary as desired to produce piezoelectric substrate tubes 12 of similar diameter. The stainless steel rod 14 may be rotated about the longitudinal axis 14 at a slow and steady rate as the extruded molten material 15 is deposited onto the stainless steel rod 14 to form a wire pattern in the form of a cylinder or tube on the stainless steel rod 14.

The 3D printing system 10 may further include a filament extruder 30 including a filament guide 31 and feeding rollers 32 that are driven by a motor to drive the filaments 34 downward through the extruder 30 and into an extrusion nozzle 24. The extrusion nozzle 24 may be heated by a heater 36, such as a thermistor, thermocouple, or the like mounted to the extruder 30 to heat the extrusion nozzle 24 to melt the filaments 34 as it flows through the extrusion nozzle 24 to dispel the extruded material 15 in molten form. The heater 36 may heat the extrusion nozzle 24 and filaments 34 to a temperature of at least 130 degrees Celsius, and at least 140 degrees Celsius, and at least 150 degrees Celsius, and at least 160 degrees Celsius, and between 150-200 degrees Celsius to cause the filaments 34 to properly melt and be properly extruded through the extrusion nozzle 24 without clogging. In one embodiment, the extruder may have a 2.85 mm diameter nozzle heating the filaments 34 to about 162 degrees Celsius.

The 3D printing system 10 may further include a printer head 41 supporting a printer nozzle 43 which may be translated in all directions but primarily along the longitudinal axis 16 above the stainless steel rod 14 to assist with building the printed object as understood in the art. The extruded material 15 is received from the extrusion nozzle 24 to the printer nozzle 43 to be expelled through a small nozzle opening 45 so that a small amount of extruded material 15 is deposited onto the outer curved surface 20 of the stainless steel rod 14. The nozzle opening 45 may have a diameter of less than or equal to 1 mm, and less than or equal to 0.5 mm, and less than or equal to 0.4 mm, and less than 0.3 mm, and less than 0.2 mm, and less than 0.1 mm, and ranging from 0.1 mm to 1 mm. The printer nozzle 43 may be heated by a heater 38, to heat the printer nozzle 43 to heat the extruded material 15 as it flows through the printer nozzle 43. The extruded material 15 may be heated to a temperature of at least 200 degrees Celsius, and at least 210 degrees Celsius, and at least 220 degrees Celsius, and at least 230 degrees Celsius, and at least 240 degrees Celsius, and between 200-250 degrees Celsius during printing. In one embodiment, the printer nozzle 43 has a nozzle opening 45 with a diameter of 0.5 mm and is heated to about 240 degrees Celsius for printing.

The printer head 41 of the 3D printing system 10 may be moved vertically up and down, for example, to position the printer nozzle 43 above the stainless steel rod 14 and translated primarily along the longitudinal axis 16 of the stainless steel rod 14 to assist with building the wire pattern of the printed object. The stainless steel rod 14 may have a length of at least 50 mm and at least 100 mm and at least 150 mm and at least 200 providing a sufficient length to build the printed object and may be generally much longer than the length of the printed object. The stainless steel rod 14 is rotated about the longitudinal axis 16 and may have a diameter between 2 to 50 mm and between 2 to 20 mm and about 6 mm and about 2.5 mm providing a sufficient outer surface area to build the printed object and is generally about the same diameter as the printed object.

The printer nozzle 43 may be used not only to melt and print the extruded material 15 but also to pole the extruded material 15 as the extruded material 15 is deposited onto the stainless steel rod 14. The printer nozzle 43 serves as an anode of the electrical field assembly and is connected to a high voltage source 39. Thus, the printer nozzle 43 may be manufactured of a heat and electrically conductive material such as brass, stainless steel, and hardened steel allowing for melting of extruded material 15 and poling processes. Similarly, the stainless steel rod 14 serves as a cathode of the electrical field assembly and is also connected to the high voltage source 39. Thus, the stainless steel rod 14 may be manufactured of a heat and electrically conductive material such as brass, stainless steel, and hardened steel allowing for poling. The printer nozzle 43 and stainless steel rod 14 thus produce a poling electric field.

The piezoelectric substrate tube 12 is printed on the outer surface 20 and about the circumference of the stainless steel rod 14 with at least one layer of extruded material 15 according to the following printing and poling process described below.

First, the outer curved surface 20 of the stainless steel rod 14 is coated with an adhesive layer 26 to assist with the adhesion of the extruded material 15 to the stainless steel rod 14. The adhesive layer 26 may be a thin layer of polyvinyl alcohol (PVA) glue, PVA hydrogel, Kapton tape, masking tape or the like enhancing the adhesion of the lowermost print layer to the stainless steel rod 14. The adhesive layer 26 may have a thickness that is less than 50 micrometers.

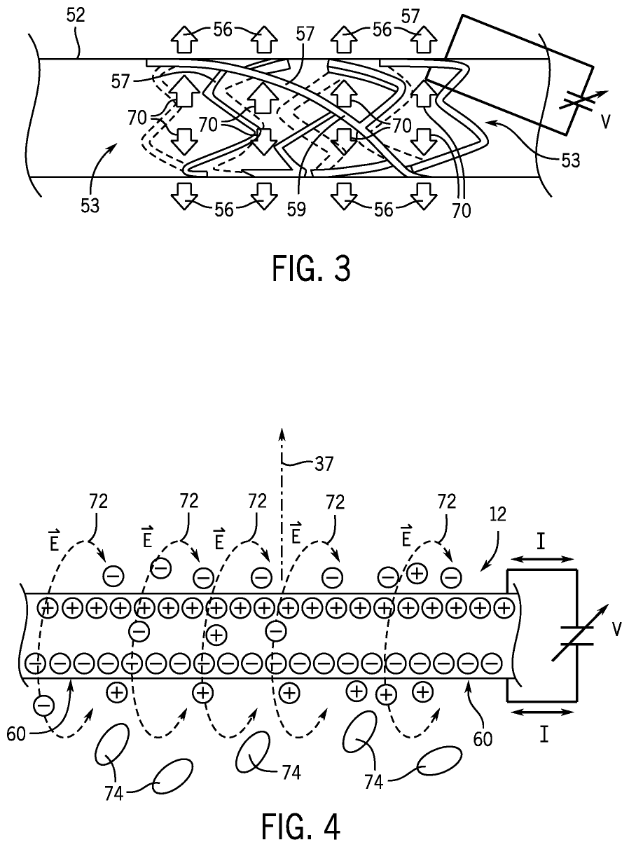

Second, the extruded material 15 is applied to the stainless steel rod 14 by the printer nozzle 43 as it moves along the longitudinal axis 16 while the stainless steel rod 14 is simultaneously rotated. As the extruded material 15 is extruded through the printer nozzle 43, a zigzag pattern is formed encompassed by, for example, five zigzag wire rings 57 each connected by diagonal wire bridges 59 as seen in FIG. 3. In one embodiment, the printer nozzle 43 moves along the longitudinal axis at 2.5 mm per second while the stainless steel rod 14 is rotated at 0.2 revolutions per second.

As the extruded material 15 is extruded through the printer nozzle 43, an electrical field is created between the stainless steel rod 14 and the printer nozzle 43 to pole the extruded material 15 as it is being extruded, causing polarization of the extruded material 15 as it is deposited onto the stainless steel rod 14. To pole the extruded material 15, an adjustable electric field in the range of 0.5 to 4 kV/mm is applied between the stainless steel rod 14 and the printer nozzle 43. Thus, the poling direction is perpendicular to the longitudinal axis 16 or axial direction.

The electric field is created by the high voltage source 39 having an output voltage of up to 30 kV (10 W). A positive electrode of the high voltage source 39 may be connected to the printer nozzle 43 while a negative electrode of the high voltage source 39 may be connected to the stainless steel rod 14. The direction of poling along a poling axis 37 or radial direction dictates the polarity of the extruded material 15, however, the anode and cathode may be switched without affecting the piezoelectric effect of the resulting printed piezoelectric substrate tube 12. The poling of the extruded material 15 causes the dipoles of the printed piezoelectric substrate tube 12 to orient in the same direction, leading to a piezoelectric effect of the material. In one embodiment, the printing nozzle 43 and stainless steel rod 14 are connected respectively to the positive and negative electrodes of the high voltage source 39 (30 kV, 10 W). Thus, the piezoelectric substate tube 12 with 6 mm internal diameter is poled simultaneously with a built-in electric field of 1.2 kV mm⁻¹ during the 3D printing process.

The electric field can be applied at elevated temperatures, for example, at least 230 degrees Celsius, and at least 250 degrees Celsius, and between 230-290 degrees Celsius, and at temperatures required to properly melt the filament 34. At these elevated temperatures, it would normally be expected that the extruded material 15 would exhibit a significantly lower coercive force, therefore requiring the extruded material 15 to cool prior to effective poling application. However, the concentrated electric field and high processing temperature are favorable for aligning the ferroelectric dipoles in the extruded material 15. Thus, the extruded material 15 is poled simultaneous with extrusion onto the stainless steel rod 14 eliminating the need for separate 3D printing and poling steps.

The extruded material 15 may be deposited onto the stainless steel rod 14 as a single layer of wire, forming the piezoelectric substrate tube 12 having a wire diameter or wall thickness of at least 0.1 and at least 0.15 mm and between 0.1 mm to 0.2 mm (100 μm to 200 μm). The piezoelectric substrate tube 12 may be printed at any desired length, e.g., 2 mm to 200 mm, and with a substantially uniform diameter, e.g., 2 to 50 mm or 2 to 20 mm. A zigzag lattice allows the piezoelectric substrate tube 12 to be compressed into smaller length and diameter for catheter delivery (by closing the gaps or openings between the lattice) and released by regular balloon expansion (by expanding the gaps or openings between the lattice) as further discussed below.

Referring to FIG. 2, in an alternative embodiment, the poling direction is in-plane of the piezoelectric substrate tube 12 instead of perpendicular to the piezoelectric substrate tube 12 as seen in FIG. 1. To achieve in-plane polarization along a poling axis 37 in an axial direction that is in-plane to the piezoelectric substrate tube 12, an interdigital electrode (IDE) 47 is prefabricated on a printing plane of a build plate 48 with laterally extending digit electrodes extending in parallel along the width (x-axis) direction or radial direction, supported by left and right digit electrodes extending along the length (y-axis) direction or axial direction, and configured to support micro-lattice printing of a planar micro-lattice substrate 49 thereon. The IDE 47 is configured to match the desired dimensions of the planar micro-lattice substrate 49 (and surface area of the piezoelectric substrate tube 12) to pole the entire surface area of the planar micro-lattice substrate 49. The planar micro-lattice substrate 49 may be printed into a zigzag pattern with zigzag wire rings 57 each connected by wire bridges 59 as seen in FIG. 3.

During the printing process, the IDE 47 is connected to a high voltage source 39 to provide an in-plane poling electric field along poling axis 37, which is adjusted to align the ferroelectric dipoles of the planar micro-lattice substrate 49 along poling axis 37 in situ. The interdigitated configuration of the IDE pattern 47 ensures that the polarity of each individual unit or each zigzag wire ring 57 of the planar micro-lattice substrate 49 is opposite to the adjacent unit or zigzag wire ring 57 along the length (y-axis) direction or axial direction. In certain embodiments, electric fields strengths between 0.5-4 kV/mm are applied to in-situ pole the planar micro-lattice substrate 49 where the planar micro-lattice substrate 49 is simultaneously printed at a range of speeds between 1-20 mm/s and at temperatures between 240-280° C. The planar micro-lattice substrate 49 is then rolled about the length (y-axis) direction or axial direction and joined at the seam by local heating to construct the piezoelectric substrate tube 12.

It is understood that alternative methods may be used to fabricate the piezoelectric tube substate 12 such as those described in U.S. Pat. No. 11,849,642, hereby incorporated by reference and assigned to the present assignees.

Referring again to FIGS. 1 and 2, the filament 34 used in the printing processes described above and which is melted to produce the piezoelectric substrate tube 12 may be a ferroelectric composite 40 formed of ferroelectric potassium sodium niobite (KNN) or KNN-based piezoceramic particles 42 embedded in a ferroelectric polyvinylidene fluoride (PVDF) or PVDF-based polymer 44. The KNN-based piezoceramic particles 42 may include KNN, Li/Sn/Ta-doped KNN, KNN-ABO3 solid solution, and the like. The PVDF-based polymer 44 may include PVDF, PVDF-TrFE, PVDF-HFP, PVDF-CTFE, other PVDF-copolymers, and the like.

The KNN-based piezoceramic particles 42 are a group of high performance, lead-free ferroelectric ceramics. The KNN-based piezoceramic particles 42 possess high piezoelectric properties and have a piezoelectric coefficient (d₃₃) of about 100 pC/N and between 80-400 pC/N at room temperature, and a high Curie temperature of at least 270 degrees Celsius and at least 300 degrees Celsius and at least 400 degrees Celsius and between 435-445 degrees Celsius and about 440 degrees Celsius, which assist the composite material in maintaining its ferroelectric phase during high temperature 3D printing. The KNN-based particles piezoceramic 42 have a high dielectric permittivity of about 200 at 10 kHz at room temperature. The KNN-based piezoceramic particles 42 may generate a stable piezoelectric peak-to-peak voltage output (Vₚₚ) of about 20V and a short-circuited current of about 30 ρA under an external force load of 30 N peak load. The KNN-based piezoceramic particles 42 are also biocompatible making them a suitable material for stents.

In certain embodiments, the KNN-based piezoceramic particles 42 may be prepared to further include 3-(trimethoxysilyl) propyl methacrylate (MPS) 46 that is covalently grafted on the outer surface of the KNN piezoceramic particles 42 to improve the interface compatibility and maximize the dispensability of the KNN piezoceramic particles 42.

The PVDF-based polymers 44 are a group of soft thermoplastic polymers with high printability, flexibility, and acceptable piezoelectricity. The PVDF-based polymers 44 possess piezoelectric properties and have a piezoelectric coefficient (d₃₃) of about 20 pC/N at room temperature, and a Curie temperature of between 190 and 200 degrees Celsius and about 195 degrees Celsius, which assist the composite material in maintaining its ferroelectric phase during high temperature 3D printing. The PVDF-based polymers 44 have a high dielectric permittivity of about 12 at 10 kHz at room temperature. The PVDF-based polymers 44 may generate a stable piezoelectric peak-to-peak voltage output (Vₚₚ) of about 3V and a short-circuited current of about 100 nA under an external force load of 30N peak load. The PVDF-based polymers 44 are also biocompatible making them a suitable material for artificial blood vessels. The PVDF-based polymers 44 have low electrical conductivity.

The process of making the ferroelectric composite 40 may be as described in U.S. Pat. No. 11,849,642 hereby incorporated by reference and assigned to the present assignees. It is also understood that other processes may be used in the manufacture of the filament 34 of ferroelectric composite 40.

The filament 34 of ferroelectric composite 40 may be used to 3D print a piezoelectric substrate tube 12 according to the methods shown and described with respect to FIGS. 1 and 2 above. The ferroelectric composite 40 contains uniform distribution of KNN piezoceramic particles 42 inside the PVDF-based polymer 44 matrix. The ferroelectric composite 40 may consist of 30-40 vol % and about 35 vol % functionalized KNN-based piezoceramic particles 42 and 60-70 vol % and about 65 vol % PVDF-based polymer 44.

The piezoelectric substrate tube 12 has a piezoelectric coefficient (d₃₃) of at least 4 pC/N and at least 5 pC/n and about 4.2 pC/N at room temperature, and a Curie temperature of at least 250 degrees Celsius. The printed piezoelectric substrate tube 12 may generate a stable piezoelectric peak-to-peak voltage output (Vₚₚ) of at least 10 mV and from 10 to 150 mV corresponding to pressure changes from at least 40 mmHg, which is the pressure change expected within the body showing a good piezoelectric output for this application.

Referring to FIG. 3, the above described method of 3D printing a piezoelectric substrate tube 12 may be used to manufacture a stent 50 with self-powered electric field generation capability within the body. In this respect, the stent 50 may also be used to produce a desirable low intensity electric field around the stent 50 within the body where the stent 50 is implanted.

Specifically, the stent 50 is a hollow cylinder or tube installed within the lumen of a blood vessel 52 or used in place of a blood vessel and has circular open ends 53 allowing blood to flow through. The piezoelectric substrate tube 12 is poled along a poling axis 37 that is normal to the plane of the substrate, as shown in FIG. 3, or alternatively be poled along a poling axis 37 that is along or in-plane with the substrate and described above with respect to FIGS. 1 and 2, respectively, to self-generate a voltage with blood vessel fluctuations 56.

The piezoelectric substrate tube 12 may be printed in a zigzag pattern 55 to allow for compression and expansion of the stent 50 within the blood vessel. The zigzag pattern 55 may comprise of a repeating pattern of zigzag rings 57 connected by bridges 59 which interconnect each of the zigzag rings 57. The positions of bridges 59 between each zigzag ring 57 are at approximately 90-degree intervals around the circumference of the zigzag rings 57. The zigzag rings 57 may have a diameter between 2 mm to 50 mm or between 2 to 10 mm or about 6 mm or about 2.5 mm. The bridges 59 may be angled at about 45 degrees between the zigzag rings 57. The inclined bridges 59 may have a total length of about 3 mm thus spacing the zigzag rings 57 apart by about 3 mm.

The printed zigzag pattern 55 of the piezoelectric substrate tube 12 reduces the surface area of the piezoelectric substrate tube 12 thus making it more flexible and providing a lower mechanical modulus. For example, the zigzag pattern 55 may include less than 30% printed area and more than 70% openings of the surface area of the piezoelectric tube 12. The increased flexibility provided by the zig zag pattern 55 allows the piezoelectric substrate tube 12 to be compressed, by closing the openings, and expanded, by opening the openings, without breaking. Thus, the piezoelectric substrate tube 12 exhibits tissue-comparable elasticity and flexibility.

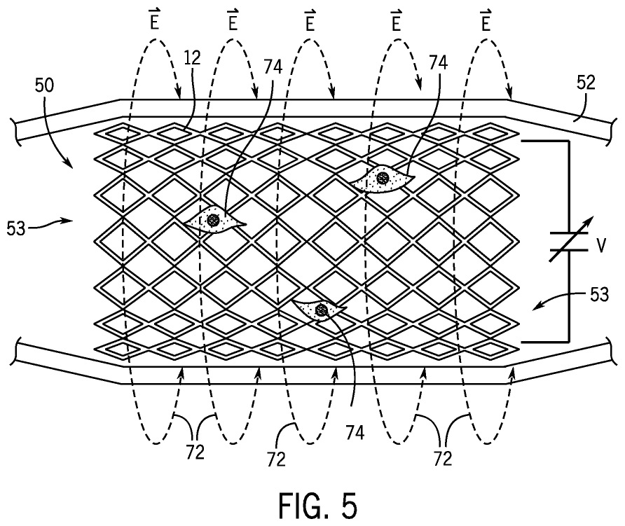

It is understood that other patterns may be employed such sinusoidal or trapezoidal rings or the bridges 59 may be omitted from the zig zag pattern 55, as seen in FIG. 5.

Referring to FIGS. 3 and 4, the mechanical changes in the piezoelectric substrate tube 12 are converted to an electrical charge which creates an electric field. For example, the mechanical changes to the piezoelectric substrate tube 12 are caused by the changes in blood flow and pressure through the inner lumen 60 of the piezoelectric substrate tube during blood vessel fluctuations 56, i.e., expansion and recoil of the blood vessel, vasodilation (widening of the blood vessels) and vasoconstriction (narrowing of the blood vessels). For example, an average adult has a diastolic pressure of 80 mmHg and a systolic pressure of 120 mmHg, and the pressure fluctuates within pressure changes around 40 mmHg (5.33 kPa). The changes in blood pressure 70 will result in a positive or negative voltage output between the inner and outer surface of the piezoelectric substrate tube 12 and along the poling axis 37 of the piezoelectric substrate tube 12. The polarization of voltage is consistent with the poling direction of the piezoelectric substrate tube 12 during the 3D printing method described above with respect to FIG. 1.

The peak-to-peak voltage output (Vₚₚ) may be at least 10 mV corresponding to the normal human blood pressure change around 40 mmHg. Thus, the voltage output generates an alternating, low intensity and discrete electric field generated by the stent 50 in response to blood pressure fluctuations 56.

As seen in FIGS. 4 and 5, as the stent 50 expands and contracts, alternating positive or negative voltage output will cause a low frequency, low intensity electric field 72 to be created to disturb the bio-film growth 74 on the stent 50 by disturbing the electrical double layer of the poled piezoelectric substrate tube 12. The low intensity electric field 72 is low strength to not harm the surrounding blood vessel cells. The low intensity electric field 72 may be less than 12 V/cm and greater than 0 but less than 12 V/cm.

The anti-biofouling and anti-biologic growth mechanisms are attributed to the electric field-induced disturbance to the electrical double layer of the poled piezoelectric substrate tube 12, which impairs the stable adsorption of organic substances and the subsequent microbe or biologic attachments.

### EXAMPLES

### Example 1: 3D-Printed Piezoelectric Stents for Electricity Generation Driven by Pressure Fluctuation

### Materials and Methods

A piezoelectric zig-zag shaped stent was printed directly on a rotating stainless-steel rod. The rod was used as a support surface for 3D printing, as well as to provide a shape template to define the size of the stent. Here, a rod with a diameter of 6 mm was used, which could produce 6 to 6.5 mm stents that fit a 6.5 mm sheath. The schematic setup is as shown in FIG. 1.

A high voltage of 1.2 kV was applied between the printer nozzle and the stainless-steel rod during the entire printing process. It introduced an in-situ poling electric field of 1.2 kV through the as-printed ferroelectric filament to align its polarization. The rotation rate (one rotation per 5 seconds) was coordinated with the nozzle movement speed and position to create a zigzagged ring pattern continuously along the axial direction. Through this approach, a stent made of piezoelectric filament could be directly printed at any selected length with a uniform diameter. The as-printed stent had an internal diameter of 6 mm composed of 5 zigzag rings giving a total length of 2.4 cm. The zigzag lattice allowed the stent to be compressed into smaller size for catheter delivery and released by regular balloon expansion.

The strong in situ poling electric field could align the randomly oriented KNN ferroelectric domains in the stent filament along a radial direction. Therefore, the radial direction was considered as the out-of-plane direction, and the generated piezoelectric potential was defined between the outer and inner surfaces of the stent. The large d₃₃ value and high curie temperature (˜440° C.) of KNN allowed the stable presence of the piezoelectric phase under the elevated temperature during printing. PVDF-HFP served as a flexible and thermostable polymer matrix to achieve a high structural integrity of the stent. It may also form the ferroelectric β-phase driven by the polarized ceramic-polymer interface (polarization) and make additional contribution to the piezoelectricity of the printed structure. The fluorine-based polymer could also offer a hydrophobicity surface that is desired for blood interaction. KNN microparticles combined with PVDF-HFP matrix to form a versatile piezoelectric stent with line-scale d₃₃ over 4.2 pC N−1.

Preparation of piezoelectric composite: The piezoelectric composite was made from ferroelectric K_{0.5}Na_{0.5}NbO₃ (KNN) and PVDF-HFP. KNN microparticles was prepared by standard solid-state reaction method, consisting of potassium carbonate (K₂CO₃, >99%, Sigma), sodium carbonate (Na₂CO₃, >99.5%, Sigma) and niobium (V) oxide (Nb₂O₅, >99.9%, Chemsavers) at a molar ratio of 1:1:2. The pile of K₂CO₃, Na₂CO₃ and Nb₂O₅ powder was mixed in a nylon mill jar including zirconia grinding balls and further grinded together with ethanol for 2 h by a gear-drive planetary ball mill machine (BM4X-V2.0L, Col-Int Tech, LLC). Afterward, the soupy mixture was dried at 60° C. in an oven overnight to dehydrate. Large lumps were removed by using a 100-mesh lab sieve from the fully dry mixture, and then crushed and grinded by mortar and pestle sets for 2 h. The subtle particles were sintered in a muffle furnace at 1100° C. for 4 h and then cooled down naturally to room temperature. Ferroelectric KNN microparticles were achieved after grinding manually for another 2 h. PVDF-HFP pellets diced from Fluorinar-C PVDF filament (NPFC175W1000, Nile Polymers, LLC) were dissolved in N,N-dimethylformamide (DMF, >99.8%, Sigma) solvent with 20 wt % solute, followed by stirring for 1 h at 80° C. Corresponding prepared KNN microparticles with 139 wt % of PVDF-HFP were added into the uniform solution and kept stirring until completely mixed. The final mixture was transferred into a fume hood to evaporate DMF solvent and then kept dry in oven at 70° C. overnight, and subsequently grinded within liquid nitrogen by a multifunctional electric grinder (GS-810N, Secura, LLC) into fine piezoelectric composite.

Extrusion of piezoelectric filament: The as-prepared KNN/PVDF-HFP composite was added into a polymer extruder (assembled from Filastruder kit) and extruded through a 2.85-mm nozzle at 162° C. The size of the extruded piezoelectric filament was assessed and controlled by a filament guide with a diameter of 2.85 mm.

3D Printing of piezoelectric stents: The piezoelectric stent was printed by a custom fused deposition modeling 3D printer (LulzBot TAZ Pro 3D Printer, Lulzbot, LLC) through a 0.5-mm printing nozzle at 240° C. A rotating stainless-steel rod with a diameter of 6 mm was driven by a 12 V gear motor and used as the supporting substrate. The surface of the rod was coated with a thin layer of PVA glue (Elmer's Disappearing Purple Glue Stick, Elmer's, LLC) to improve the adhesion of printed materials. The head nozzle kept printing and moving back and forward along axial direction at 2.5 mm per second, meanwhile with supporting rod rotating on site at 0.2 revolutions per second. With the simultaneous motion of printing nozzle and rotating rod, a zigzag shaped piczoclectric stent including five rings connected by an inclined bridge was printed out. The printing nozzle and rotating stainless-steel rod were connected respectively to the positive and negative electrodes of a high voltage source (30 kV, 10 W) during the entire printing process. The piezoelectric stent with 6 mm internal diameter was poled simultaneously with a built-in electric field of 1.2 kV mm⁻¹ during 3D printing process.

Morphology, structure and piezoelectric characterization: The surface morphology was captured by a fluorescence LED microscope (Leica DM4 M) and a Zeiss Gemini 450 field-emission scanning electron microscope (SEM). X-ray diffraction (XRD) patterns of piezoelectric composite were characterized by a Bruker D8 Discovery X-ray powder diffractometer with 2-d detector. Ferroelectric property of piezoelectric composite, represented by a square layer (0.5 cm×0.5 cm) of printed piezoelectric thin film, was measured by a polarization-electric field (P-E) hysteresis loop conducted by a P-E loop workstation (Premier II, Radiant Technologies Inc., Albuquerque, NM, USA). PFM amplitude and phase characterizations were performed on a printed thread with 0.5 mm in diameter using an XE-70 Park System. The piezoelectric coefficients d₃₃ of printed thread with 0.5 mm in diameter were obtained by a quasistatic d₃₃ piezometer (ZJ-3A, Institute of Acoustics, China). All compressive and tensile modulus tests of printed stents including cyclic compression-release were measured by a Rheometrics Solids Analyzer III dynamic mechanical analyzer.

Evaluation of biocompatibility: Cell Morphology Examination. UV-sterilized samples of the P(VDF-HFP)-KNN were placed on the inside of the transwell insert of a 24-well plate with clear walls and bottom (Corning 3422). No material was added to the control wells. There were four wells in each group. In total, 1.66×103 MOVAS cells were seeded into each well containing 700 μL of cell culture medium. After 24, 48 and 72 h, the cytoskeleton and nucleus of the cells were stained with Alexa Fluor 555 Phalloidin (Thermo Fisher Scientific, A34055) and DAPI, respectively. After staining, the cells were imaged using a Keyence BZ-X800 fluorescence microscope.

Biocompatibility evaluation: MOVAS cells were purchased from American Type Culture Collection (ATCC, CRL-2797) and grown as recommended in modified DMEM containing 4.5 g L-1 D-glucose (Thermo Scientific, 11965118) supplemented with 10% fetal bovine serum (FBS), 100 U mL-1 penicillin, and 100 UmL-1 streptomycin. The cell biocompatibility of the P(VDF-HFP)-KNN was assessed with a CellTiter-Glo assay (Promega, G9242) as described. UV-sterilized samples of the P(VDF-HFP)-KNN were placed inside of the transwell insert of a 24-well plate with clear walls and bottom (Corning 3422). No material was added to the control wells. There were three wells in each group. In total, 1.66×10⁴ cells were seeded into each well containing 700 μL of cell culture medium. After incubation for 24, 48 and 72 h at 37° C. and 5% CO₂, the cell culture medium was aspirated and 350 μL of PBS and 350 μL of CellTiter-Glo solution were added to each well followed by incubation at room temperature for 30 min. Luminescence was recorded on a Varioskan LUX microplate reader (Thermo Scientific). The relative cell viability was expressed as (luminescence of sample wellsblank) (luminescence of control wells-blank)×100%, where blank is the luminescence of the wells without cells (PBS and CellTiter-Glo solution only).

Piezoelectric output measurements: The printed stents were settled tightly on a gold-coated polydimethylsiloxane (PDMS) hollow tube with an external diameter of 6 mm. Another layer of PDMS was applied to fill the surficial space to avoid short circuit, followed by a second layer of gold (50 nm in thickness) using e-beam evaporation. Two gold threads were subsequently attached to the first and second layer of gold electrode, respectively. A balloon system (Cordis, Corporation, USA) replete with flowing fluids was inserted into the PDMS hollow tube to puff up the whole device. A syringe pump affiliated with the balloon was loaded with a pressure gauge ranging from 0 to 440 Psi to control and monitor the internal fluid pressure. At the same time, the syringe pump was driven by the up-and-down motion of a computer-controlled actuator (LinMot) to 5 mm, 10 mm, 12 mm and 15 mm, corresponding to 20 Psi, 70 Psi, 90 Psi and 120 Psi. The piezoelectric voltage output of the fabricated device was recorded by a multimeter (DMM 6500, Keithley) and filtered by Band block FFT off 59.9-60.1 Hz. The long-term stability of the piezoelectric performance was further evaluated by an electrometer (6515 System, Keithley).

### Results and Discussion

The morphology of as-printed stent filament was first observed by an optical microscope. The stent filament had a uniform thickness of ˜250 μm printed through a 0.25-mm nozzle. The stent surface was smooth, and no abrupt surface extrusion or defects could be observed. The cloudy gray dots throughout the filament body indicated a uniform distribution of the KNN microparticles in the PVDF-HFP matrix. The uniform mixture of the KNN-PVDF-HFP composite was further visualized by scanning electron microscopy (SEM) image. The as-printed surface had a rather low surface profile and did not show any feature of particle agglomerations. Higher resolution SEM image clearly revealed well-proportioned distribution of largely imbedded KNN microparticles with a cubic shape and a size of approximately 1-2 μm. This homogeneous mixture is important to ensure uniform piezoelectric responses and good mechanical integrity.

X-ray diffraction (XRD) characterization was then used to confirm the presence of the ferroelectric phases in the as-printed composite filament. The XRD spectrum revealed two sets of diffraction peaks. The strong 2θ peaks near 22.8°, 31.8°, 46.9° and 52.2°, according to JCPDS card 77-0038, were from the ferroelectric KNN microparticles. One more small peak at 20.8° was also observed corresponding to the β phase of PVDF-HFP, suggesting that a small portion of PVDF-HFP was crystalized into its ferroelectric phase possibly due to the interface influence. The ferroelectric property of the as-printed composite (in a thin film morphology) was evaluated by polarization-electric field (P-E) hysteresis loop. For a 0.5-mm thick film, the polarization could reach a saturation point as high as ˜1.75 μC cm⁻² at an electric field of ˜6 kV/mm. The remanent polarization at zero electric field also had an appreciable value of ˜0.56 μC cm⁻² suggesting it may serve as a good piezoelectric building block for mechanoelectrical coupling.

The localized piezoelectric property was mapped from the as-printed piezoelectric filament surface by piezoresponse force microscopy (PFM) to reveal the distribution of surface piezoelectric polarization at the sub-micron scale. PFM characterizations were conducted on samples with and without applying the electric field during printing to confirm the effect of in situ poling. For the poled configuration, a strong amplitude displacement was shown by the bright intensities approaching ˜100 mV. Furthermore, in the phase analysis it was observed a uniform phase contrast which indicated a uniform dipole alignment of ˜180° across the surface of the poled sample. For the unpoled configuration, the amplitude response only reached a magnitude of ˜80 mV and the phase contrast were less uniform across the surface. The surface topography of samples with and without in-situ poling were also presented. The surface topography on the poled sample was overlayed with its amplitude image and revealed a vertical morphology variation of ˜200 nm and amplitude response of ˜100 mV. In contrast, the topography image without poling exhibited surface fluctuation of ˜400 nm but lower amplitude response of ˜80 mV. Although with lower topography variation, the poled sample still showed larger amplitude response compared to unpoled sample. Together, this highlights the importance of the applied electric field during the printing process.

As the essential function of a stent is to provide mechanical support in blood vessels, the mechanical properties of as-printed stents were evaluated comprehensively. First, the compressive and tensile behaviors were investigated by introducing linear deformation to the stent along its radial direction. In the compressive test, the stress-strain curve showed an almost linear relationship at the first ˜30% strain. The slope within this range yielded a compressive modulus of 37696.5 Pa. The stent exhibited a quicker yield as the strain exceeded 35%, while reaching a failure point at 69.9% strain. The tensile performance was characterized by introducing a pair of internal bars inside the stent to introduce uniform tensile stress around the entire length of stent. Almost the entire stress-strain curve showed a good linearity with a slope of 31993.2 (from 5-75% strain), corresponding to the stent's tensile modulus. The stent failed at 78.7% tensile strain. Thereby, the on-site stent in blood vessels is capable of facing dynamic blood flow and scour, exhibiting valuable functionality and strength for stent deployment. And the maximum of both compressive strain (of ˜70%) and tensile strain (of ˜79%) revealed desirable feasibility compared to most stents and blood vessels.

The strain-related mechanical properties were then evaluated from strain-release cycles at a series of strain rates ranging from 5% to 30%. At different compressive strain ranges, the stent showed a close modulus of 39930 Pa at lower strains of 5%-20%. A typical hysteresis could be observed from each cycle, suggesting the strain softening phenomenon of heterogeneous materials. Evaluated stents kept slight but acceptable deterioration until 30% compressive strain, which still overmatched the strain of blood vessels. Similar strain-related cycling behavior was observed from the series of tensile strains from 5% to 30%. With increasing tensile strain from 0 to 30%, slope of curves tilted, corresponding to decreased tensile strength, revealing subtle deformation and softening hysteresis.

Finally, the cycling behavior of the stents was tested to evaluate its long-term performance under constant compressive straining actions, representing the practical stent application situations. Considering the intimal average circumferential compressive strain of stent was up to 13.1% and that of physiological blood vessels was 4.8%, a maximum 20% compressive strain was chosen in the cycling tests at a constant strain rate. The cycling strain-stress curves showed a perfect match from continuous 20 strain cycles, suggesting the stent could sustain its stable mechanical property when compressed repeatedly to its maximum strain range. The long-term application potential of the stent was evaluated by performing the same cycling test over a much longer period. The strain-stress cyclic curves were measured at different cycling numbers from 0 to 100K after one-day relaxation. All the curves showed reliable flexibility and stability with compressive modulus from 552591.1 Pa to 56519.3 Pa within 15% strain. Better compressive strength compared to previous data was due to slightly decreased diameter from 7.00 mm to 6.55 mm after cyclic compression.

Therefore, 3D printed stents can be applied in the position of a regular stent to provide mechanical support to blood vessels.

The KNN/PVDF-HFP was designed to act as a formulated composite to serve as a novel stent material to be implanted in the vascular system and therefore, their biocompatibility was evaluated. The biocompatibility of the KNN/PVDF-HFP was first examined by analyzing mouse aortic smooth muscle (MOVAS) cells incubated with the stent material and comparing them to cells without any exposure (control group). Immunofluorescence staining images revealed that cells incubated with KNN/PVDF-HFP for 24, 48 and 72 hours exhibited the expected elongated morphology, along with appropriate distributions and densities. Importantly, there were no discernible differences compared to the cells in the control group. Additionally, a CellTiter-Glo (CTG) assay was conducted to quantitatively measure the viability of MOVAS cells in contact with the material. The results indicated that over the different time points the average cell viability of KNN/PVDF-HFP was 104.7%, showing no significant deviation from the control groups, which exhibited 100% viability. These findings affirm that the KNN/PVDF-HFP stent material is nontoxic and biocompatible.

The piezoelectric output from the stents was eventually characterized under controlled strains in simulated pressurized conditions. In the setup, a stent was sheathed on a flexible and gold coated PDMS tube. The gold surface (connecting the inner surface of the stent) was covered by an additional layer of insulating PDMS, and another gold layer was coated on the outer surface of the stent. Electrical output was measured from these two gold layers, representing the piezoelectric potential between the inner and outer surfaces of the stent. The stent-loaded and packaged PDMS tube was connected to a syringe pump integrated with a computer-controlled actuator, which was utilized to control the internal pressure at a designed rate. The internal pressure change could drive the expansion and recovery of the PDMS tube, which could induce subtle strains to the stent simulating the movements in response to blood pressure fluctuations. Under a constant fluid pressure of 20 Psi, the stent produced a stable voltage output with a peak-to-peak value of ˜0.07 V, followed by a zoom-in image of single peak. The electric outputs were in good accordance with the pressure difference (ΔP) and increased monotonically as the pressure difference increased. As ΔP increased from 20 to 120 Psi, the voltage output remained a stable pattern indicating a reliable piezoelectric performance of the stent. The average peak-to-peak voltage increased from 0.07 V to 0.15 V, suggesting the potential to sensing blood pressure change. The higher voltage peaks produced by forward electrode connection, compared to that by reverse electrode connection, was possibly a result of the thick PDMS tube wall, which might exhibit slower rate for shape recovery as the pressure reduced to the baseline. Long-term stability of the piezoelectric performance was further evaluated by measuring the electric outputs after the stent was strained repeatedly after 10000 cycles. The electric potential output measured every 2.5 k cycle was maintained within a small range from 0.25 V to 0.21 V over 10,000 straining cycles, revealing a stable piezoelectric function of the piezoelectric stent.

In summary, an intricate zigzag shaped piezoelectric stent was fabricated by FDM 3D printing assisted by in situ poling and rotation. The stent was made from a composite of ferroelectric KNN microparticles in a PVDF-HFP polymer matrix. Owing to the good homogeneity of the KNN/PVDF-HFP mixture, the as-printed stents demonstrated desired mechanical property, acceptable biocompatibility, and promising piezoelectricity. The outstanding mechanical resilience with compressive modulus of ˜37696.5 Pa and tensile modulus of ˜31993.2 Pa, together with excellent long-time compression-release stability after 100K cycles also offered a valuable avenue for in-vivo deployment. Cell viability measurement verified that there was no significant difference between control group and printed thin disc, confirming the biocompatibility and capability of piezoelectric stents. Using a simulated pressurized system, the piezoelectric voltage outputs were measured directly from the inner and outer surfaces of the stent in response to internal pressure changes. The voltage output showed good accordance to pressure variations as well as a stable output over long-term straining actions.

This development demonstrated a successful strategy for introducing an additional electromechanical coupling function to stents for in vivo blood pressure monitoring or electricity generation solely from local blood pressure variations in blood vessels. This function open possibilities of self-powered electrical stimulations to blood vessels from implanted stents, for example, for wound healing or thrombosis prevention as further described in Example 2.

### Example 2: Development of 3D-Printed Piezoelectric Stents with Self-Powered Anti-Restenosis Properties

### Materials and Methods

Printable ferroelectric bio composites consisting of high Tc (Curie temperature) ferroelectric particles (e.g., potassium sodium niobate, KNN) and thermoplastic bio-polymer matrix (e.g., Polylactic acid, PLA) are used as the printing material for stent fabrication. An electric-field-assisted 3D printing technique is employed to fabricate a planar zigzag micro-lattice structure with selectively oriented ferroelectric dipoles along the length (y-axis) direction, as shown in FIG. 2.

In order to achieve in-plane polarization, an interdigital electrode (IDE) pattern is prefabricated on the printing plate to support micro-lattice printing. The IDE is configured to match the dimensions of the stent by the following relations: IDE finger space a1=stent ring height b1, finger width a2=ring space b2, finger length a3≥stent perimeter b3; and IDE length a4≥stent length b4. During the printing process, the IDE is connected to a high voltage source to provide an in-plane poling electric field, which is adjusted to align the ferroelectric dipoles of micro-lattices in situ. The interdigitated configuration ensures that the polarity of each individual unit is opposite to the adjacent unit along the length (y-) direction. A series of electric fields with different strengths (0.5-4 kV/mm) is applied to in-situ pole the structure printed at a range of speeds (1-20 mm/s) and temperatures (240-280° C.).

After the fundamental property quantification, a stent is formed by rolling the planar microlattice with optimal mechanical and piezoelectric properties into a tubular structure and permanently joining the seam by local heating above its glass transition temperature. The mechanical stability is tested again by cyclically straining to the maximum displacement over 10 million times at a high frequency. The d₃₃ is measured again from the stent surface by PFM and compared to the value prior to the stress test. <5% d₃₃ dropping is expected given the stable ceramic ferroelectric component.

### Results and Discussion

Characterization of 3D printed stents: The piezoelectric and mechanical properties of as-printed micro-lattices are characterized to identify the optimal combination of printing conditions that gives the highest printing fidelity (mechanical stability) and piezoelectric property. Piezoresponse force microscope (PFM) is used to characterize the in-plane piezoelectricity (d₃₃) by scanning the PFM tip across all lattice units. This characterization is conducted over the entire printed structure to map the d₃₃ distribution and to ensure uniform piezoelectric property and thus uniform electricity generation. In parallel, the mechanical properties including tensile modulus, compressive modulus, flexural strength, and elongation at break are quantified by standard electromechanical test systems. The mechanical stability is tested by continuously stretching the micro-lattices along the length (y-) and width (x-) directions over 10 million times at a high frequency using a computer-controlled actuator, and the piezoelectricity mapped afterward. This high stress-test validates that the longevity of the microlattices exceeds the expected implantation lifetime.

Investigate electric field-induced inhibition of restenosis: The stent microlattices are made with biocompatible materials. In the absence of an electric field, smooth muscle cells (SMCs) are expected to attach to the stent surface and subsequently proliferate. As such, it is expected that the number of SMCs attached to stents increases with the incubation time.

In contrast, the presence of electric field prevents SMCs from attaching onto the microlattices and therefore failing to survive or expand. The correlation between the number of attached SMCs and the electric field strength is used to identify the optimal electric field. Such an electric field strength relationship provides for the optimization of the microlattice geometry.

Test the influences on endothelial repair: Proliferation and migration of endothelial cells are essential to the repair of endothelium that may sustain mechanical injuries during stent implantation. Because an intact endothelium inhibits SMC proliferation, timely repair of injured endothelium prevents restenosis from becoming a prolonged process. The printed piezoelectric microlattices do not produce adverse effects on endothelial repair.

Furthermore, the electric field from a nanogenerator may accelerate human skin wound healing and minimize scar formation. Although endothelial cells are different from skin fibroblasts in multiple aspects, a similar acceleration of endothelial repair may be induced by the electric field from the piezoelectric stents.

The quantification of endothelial wound repair is correlated to the electric field strength and used to identify the optimal electric field strength. The electric field relationships guide the design of piezoelectric stent with the most suitable electric field generation.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties.

To aid the Patent Office and any readers of any patent issued on this application in interpreting the claims appended hereto, applicants wish to note that they do not intend any of the appended claims or claim elements to invoke 35 U.S.C. 112 (f) unless the words “means for” or “step for” are explicitly used in the particular claim.

What we claim is:

1. A stent comprising:
a tube defined by
a piezoelectric substrate material formed in a cylindrical lattice pattern permitting compression and expansion of the tube;
wherein the piezoelectric substrate is poled with respect to at least one axis of the tube; and
wherein the piezoelectric substrate generates a voltage of at least 10 mV under pressure changes on an inside or outside of the tube to create an electric field surrounding the tube.

2. The stent of claim 1 wherein the piezoelectric substrate generates a voltage output of 10-150 mV.

3. The stent of claim 1 wherein the piezoelectric substrate generates a voltage output of at least 10 mV under pressure changes of at least 40 mmHg on an inside or outside of the tube.

4. The stent of claim 1 wherein the electric field is less than 12 V/cm.

5. The stent of claim 1 wherein the piezoelectric substrate is made of ferroelectric potassium sodium niobite (KNN) particles embedded in a ferroelectric polyvinylidene fluoride (PVDF) polymer matrix.

6. The stent of claim 1 wherein the substrate is poled in a radial direction or in an axial direction of the tube.

7. The stent of claim 1 wherein the tube has a uniform thickness of less than or equal to 250 μm.

8. The stent of claim 1 wherein the tube has a diameter between 2 to 50 mm.

9. The stent of claim 1 wherein the piezoelectric substrate has a length between 2 to 200 mm.

10. The stent of claim 1 wherein the cylindrical lattice pattern is zigzag rings formed about the axis of the tube joined by bridges.

11. A method of manufacturing a stent comprising a tube defined by a substrate of piezoelectric material formed in a cylindrical lattice permitting compression and expansion of a diameter of the tube wherein the substrate is poled with respect to at least one axis of the substrate, the method comprising the steps of:
heating a composite piezoelectric material at a temperature of at least 250 degrees Celsius to form a molten material;
extruding the molten material through a 3D printer nozzle;
applying an electrical field between the 3D printer nozzle and a stainless steel rod to pole the molten material as the molten material is being extruded;
depositing the molten material onto the stainless steel rod along an axis of the rod; and
rotating the rod as the molten material is deposited to form the substrate of piezoelectric material into a tube.

12. The method of claim 11 wherein the stainless steel rod has a diameter between 2 mm to 50 mm.

13. The method of claim 11 further comprising rotating the stainless steel rod once every 5 seconds.

14. The method of claim 11 wherein the molten material is deposited at a print speed of less than 20 mm/s.

15. The method of claim 11 further comprising applying an adhesive layer to the stainless steel rod prior to depositing the molten material onto the stainless steel rod.

16. A method of stenting an anatomical vessel to prevent restenosis, the method comprising the steps of:
providing a stent defined by a tube having a substrate of piezoelectric material formed in a cylindrical lattice permitting compression and expansion of the tube wherein the substrate is poled with respect to at least one axis of the substrate;
inserting the stent into a lumen of the anatomical vessel; and
generating a peak-to-peak voltage output in response to anatomical vessel fluctuations of at least 10 mV under pressure changes on an inside or outside of the stent to create an electric field around the stent.

17. The method of claim 16 further comprising creating an alternating positive or negative voltage output based on the anatomical vessel and pressure fluctuations.

18. The method of claim 16 further comprising creating an alternating electrical field based on the anatomical vessel and pressure fluctuations.

19. The method of claim 16 wherein the electric field strength is less than 12 V/cm.

20. The method of claim 16 further comprising disturbing inner and outer surfaces of the tube to prevent biologic adherence and growth.
